(19) United States
(12) Patent Application Publication
(10) Pub. No.: US 2004/0028661 A1
(43) Pub. Date: Feb. 12, 2004

(54) Expansion of cells using thrombopoietin and anti-transforming growth factor-beta

(76) Inventor: Stephen H. Bartelmez, Seattle, WA (US)

Correspondence Address: CLARK & ELBING LLP, 101 FEDERAL STREET, BOSTON, MA, 02110, US

(21) Appl. No.: 10/213,957

(22) Filed: Aug. 7, 2002

(51) Int. Cl. A61K 48/00; C12N 5/08

(52) U.S. Cl. 424/93.21; 435/366; 514/44

(57) ABSTRACT

The invention features a method for the expansion of hematopoietic stem cells using a combination of a thrombopoietin agonist and a transforming growth factor-beta blocking agent in the absence of stem cell factor. The invention also features a hematopoictic stem cell composition that has been expanded using a combination of a thrombopoietin agonist and a transforming growth factor-beta blocking agent in the absence of stem cell factor, as well as methods of using an expanded hematopoietic stem cell composition to restore or supplement an immune system and/or blood forming system compromised by, for example, radiation or chemotherapy.

### STATEMENT AS TO FEDERALLY SPONSORED RESEARCH

This research has been sponsored in part by NIH grant number R01DK48708. The government has certain rights to the invention.

### BACKGROUND OF THE INVENTION

The invention relates to expansion of relatively undifferentiated cells.

The origin of all the cells in blood and in the immune system is the hematopoietic stem cell (HSC). Each HSC has the potential to differentiate into at least eight separate blood cell lineages within the myeloid and lymphoid blood cell compartments. It has been estimated through successive generational analysis that one HSC has the potential to produce millions of differentiated progeny each day for the lifetime of the animal.

This enormous potential could be exploited if, starting from a small number of HSCs, a large pool of HSCs could be produced in an ex vivo expansion system. This pool of HSCs could then be used to restore or supplement an immune system and/or blood forming system compromised by, e.g., radiation or chemotherapy, and as a valuable tool in the design, development and testing of diagnostic and therapeutic agents used in the treatment of immune system and/or blood forming disorders.

Efforts have been made to develop systems that would promote growth of HSCs ex vivo and control cell proliferation and differentiation. Typically, these efforts have involved batch culture of a mixed population of cells which have been initially separated from a large volume of bone marrow or blood. However, there is presently no clinically approved method to preserve and expand HSCs, particularly at their earliest, most multi-potential stages. The most common approach to ex vivo multi-potential hematopoietic cell expansion is to culture purified HSCs (i.e., those expressing the CD34 marker) in the presence of early-acting cytokines, such as IL-3, stem cell factor (SCF), and IL-6. Unfortunately, using prior art cell expansion methods, there is generally an inverse relationship between cell proliferation and the percentage of primitive stem cells that are maintained in a relatively non-differentiated stage of development. Thus, using prior art methods, when HSCs are induced to proliferate, functional HSCs are lost due to differentiation. Because these early cells can be the most useful for inducing hematopoietic engraftment (i.e., in that survival of these cells in vivo provide a source for many or all blood cell types), differentiation associated with prior art cell expansion methods represents a significant shortcoming of those methods. Therefore, a significant need remains for methods that promote the proliferation of multi-potential HSCs in a manner that maintains their innate engraftment capacity and multipotentiality.

### SUMMARY OF THE INVENTION

The present invention features a method for promoting the expansion of a desired, relatively undifferentiated population of cells (e.g., HSC or long-term repopulating hematopoietic stem cells (LTR-HSC)) present in a starting population of cells, by culturing such a starting population of cells in a culture medium lacking exogenously added stem cell factor (SCF) and containing an exogenously added thrombopoietin (TPO) agonist under conditions that cause blockade of the transforming growth factor (TGF)-beta pathway. In a desired embodiment, the cells are of mammalian origin (e.g., human origin). In several desired embodiments, the TPO agonist is TPO (e.g., full length TPO and fragments thereof), more desirably, human TPO, and most desirably, recombinant human TPO. In other embodiments, the TPO agonist is a chimeric TPO (e.g., a chimeric TPO polypeptide comprising a fragment of TPO and another growth factor), a TPO analog, or a TPO peptide mimetic. In several embodiments, the TPO agonist is a molecule that can bind to and activate the myeloproliferative leukemia (mpl) receptor (e.g., an antibody (see e.g., Deng et al., Blood 92:1981-1988, 1998; Abe et al., Immunol. Lett. 61:73-78, 1998; U.S. Pat. No. 6,342,220; and U.S. Pat. No. 5,989,538); or is a molecule that promotes formation of a dimerized mpl receptor, or is a dimerized mpl receptor (see, e.g., Alexander et al., Stem Cells 1:124-132, 1996) such that mpl receptor-mediated biological activity is initiated. The TPO agonist can also be a molecule that is involved in, or can activate, the cell signaling pathway of the mpl receptor (e.g., the Janus kinase pathway). In other embodiments, the TPO agonist is a molecule that functions in the cell signaling pathway of the mpl receptor (e.g., members of the Janus kinase family, such as JAK1, JAK2, JAK3, and TYK2, a She protein, the insulin receptor substrates (e.g., IRS1, 2, and 3), and the signal transducers and activators of transcription (STATs; e.g., STAT1, STAT3, STAT5a, and STAT5b).

In other desired embodiments, blockade of the TGF-beta pathway is effected by an exogenously added TGF-beta blocking agent. A TGF-beta blocking agent is characterized by the ability to prevent TGF-beta receptor-mediated biological activity. This can be accomplished in several ways, including but not limited to, blocking binding of TGF-beta to its cognate receptor or preventing or reducing TGF-beta receptor-mediated cell signaling pathways. Exemplary TGF-beta blocking agents of the invention are identified as a neutralizing anti-TGF-beta antibody (e.g., 1D11), a TGF-beta antisense nucleic acid (e.g., antisense RNA) or peptide nucleic acid (PNA), or a soluble TGF-beta receptor. In several embodiments the TGF-beta blocking agent is an inactive TGF-beta polypeptide, or fragment thereof, a TGF-beta polypeptide analog, or a TGF-beta peptide mimetic, which is capable of binding the receptor, but not activating it. In other desired embodiments, a TGF-beta blocking agents is an antagonist of the TGF-beta receptor signaling pathway (i.e., an inhibitor that prevents or reduces signal transduction by the TGF-beta receptor). A TGF-beta blocking agent that acts as an antagonist of TGF-beta receptor signaling is a molecule that prevents (i.e., inhibits) or reduces signal transduction by the TGF-beta receptor by inhibiting the Smad signaling pathway (e.g., by inhibiting Smad2, Smad3, or Smad4). In other desired embodiments, a TGF-beta blocking agent is fibromodulin, decorin, biglycan, lumican, PG-Lb, keratocan, mimecan, EVI-1, SKI, or SNO, which repress TGF-beta signal transduction. In another desired embodiment, blockade of the TGF-beta pathway is by a molecule that inactivates a protease responsible for activating a precursor TGF-beta polypeptide into an active, mature TGF-beta polypeptide.

In other embodiments, the cells of the invention are hematopoietic cells (e.g., long-term repopulating hematopoietic stem cells). In other desired embodiments, the cells of the invention are mammalian cells (e.g., human, murine, or porcine) that are derived from, for example, bone marrow, both adult and fetal, mobilized peripheral blood (MPB) and umbilical cord blood; and respond to a culture medium containing the combination of an exogenously added TPO agonist and an exogenously added TGF-beta antagonist in the absence of exogenously added SCF.

In a desired embodiment, the method of the invention further includes culturing the cells in the presence of one or more compounds selected from a growth factor and a cytokine during and/or after exposure to an exogenously added TPO agonist and an exogenously added TGF-beta antagonist in the absence of exogenously added SCF, after expansion of the cells. Desired growth factors are selected from Flt3, SCF, VEGF, FGF, and EGF, and desired cytokines are selected from IL-3, IL-6, IL-11, and IL-12. In other desired embodiments, the cells are cultured in the presence of an exogenously added TPO agonist and an exogenously added TGF-beta blocking agent in the absence of exogenously added SCF for 2 to 4 hours, desirably 1 to 5 days, more desirably 1 to 100 days, and most desirably 1 month to 10 years.

Another aspect of the invention features a relatively undifferentiated cell that has been expanded by culturing the cell in the presence of an exogenously added TPO agonist (e.g., TPO) and in the absence of exogenously added stem cell factor, and under conditions that result in the blockade of the TGF-beta pathway (e.g., by using an exogenously added TGF-beta blocking agent).

The invention also features a relatively undifferentiated cell that has been expanded by culturing the cell in a culture medium lacking exogenously added stem cell factor, under conditions that promote activation of the TPO pathway and cause blockade of the TGF-beta pathway (e.g., by using an exogenously added TGF-beta blocking agent).

Still another aspect of the invention features a method of restoring or supplementing a compromised immune system or a blood forming system in a subject in need thereof, in which the subject is administered cells that have been expanded by culturing the cell(s) in the presence of an exogenously added TPO agonist (e.g., TPO) and in the absence of exogenously added stem cell factor, and under conditions that result in the blockade of the TGF-beta pathway (e.g., by using an exogenously added TGF-beta blocking agent).

Yet another aspect of the invention features a method of restoring or supplementing a compromised immune system or blood forming system in a subject in need thereof, in which the subject is administered cells that have been expanded by culturing the cell(s) under conditions that result in the activation of the TPO receptor signaling pathway and in the absence of exogenously added stem cell factor, and under conditions that result in the blockade of the TGF-beta pathway (e.g., by using an exogenously added TGF-beta blocking agent).

In desired embodiments of the method of restoring or supplementing a compromised immune system or blood forming system, the immune system has been compromised by chemotherapy or radiation treatment. In other embodiments, the subject is a mammal (e.g., a human). In yet other embodiments, cells are administered to the subject by transplantation. In still other embodiments, the cells have also been genetically modified to express one or more polypeptides.

One advantage of the invention is that stem cells cultured in the presence of an exogenously added TPO agonist and an exogenously added TGF-beta blocking agent in the absence of exogenously added SCF, retain the ability to undergo substantial self-renewal and proliferation without differentiation, while maintaining the ability to, at a later time, differentiate into cells of all the hematopoietic lineages, i.e., pluripotent hematopoietic stem cells.

Definitions

By “TPO agonist” is meant any molecule that is capable of activating the mpl receptor, or is capable of activating the mpl receptor signal transduction pathway. Exemplary TPO agonists include, but are not limited to, full length thrombopoietin, and fragments, thereof, a chimeric thrombopoietin polypeptide, a thrombopoietin-like molecule (e.g., thrombopoietin analogs and thrombopoietin peptide mimetics), an antibody that binds to and activates the mpl receptor, a molecule that functions in the cell signaling pathway of the mpl receptor (e.g., members of the Janus kinase family, such as JAK1, JAK2, JAK3, and TYK2, a Shc protein, the insulin receptor substrates (e.g., IRS1, 2, and 3), and the signal transducers and activators of transcription (STATs; e.g., STAT1, STAT3, STAT5a, and STAT5b). Desirably, a TPO agonist is capable of modulating (e.g., increasing) the signaling of the mpl receptor through the mpl receptor signal transduction pathway by at least about 10%, 20%, 30%, 40%, 50%, or 60%, more desirably about 70%, 80%, 90%, 95%, or 99%, or most desirably by about 100%, 200%, 500%, or 1000% or more.

By “TGF-beta blocking agent” is meant any molecule that is capable of reducing or inhibiting the activity of TGF-beta including, but not limited to, the binding of TGF-beta to its cognate receptor and activating the TGF-beta receptor signal transduction pathway. Exemplary TGF-beta blocking agents include, but are not limited to, a neutralizing antibody capable of binding TGF-beta or the TGF-beta receptor; a nucleic acid or peptide nucleic acid (PNA) molecule antisense to TGF-beta, the TGF-beta receptor, a soluble TGF-beta receptor capable of binding TGF-beta, a factor involved in TGF-beta or TGF-beta type I and II receptor subunit upregulation, or a factor involved in downstream signaling of the TGF-beta receptor (e.g., the Smad2, Smad3, and Smad4, tissue transglutaminase, Rb-1, p15, p21, and p27); a TGF-beta polypeptide, a TGF-beta analog, a TGF-beta peptide mimetic, or a polypeptide that is closely related or unrelated to TGF-beta that is capable of binding the TGF-beta receptor, but not activating the signal transduction pathway; and a polypeptide that blocks the antiproliferative effects of TGF-beta (e.g., decorin, biglycan, EVI-1, SKI, or SNO). Desirably, a TGF-beta blocking agent is capable of modulating (e.g., decreasing) the binding of a TGF-beta polypeptide to the TGF-beta receptor, or is capable of modulating (e.g., decreasing) the signaling of the TGF-beta receptor through the TGF-beta receptor signal transduction pathway, by at least about 10%, 20%, or 30%, more desirably about 40%, 50%, 60%, or 70%, or most desirably by about 80%, 90%, 95%, or 99% or more.

By “mpl ligand” is meant a compound capable of binding to the mpl receptor such that one or more mpl-mediated biological actions are initiated. Herein, the term “mpl ligand” will be used generically to refer to all polypeptides that activate the mpl receptor, including TPO and megakaryocyte growth and development factor (MGDF). The term “mpl ligand” can refer to the full length TPO polypeptide, or fragments thereof, chimeric TPO polypeptides, or TPO polypeptide analogs. As herein disclosed, mpl-mediated biological activity includes (1) promotion of the survival of stem cells in culture, such that the cell maintains the capability of self-renewal and the ability to give rise to all hematopoietic cell lineages, (2) expansion of stem cell populations, such that the expanded cell population maintains the capability of self-renewal and the ability to give rise to all hematopoietic cell lineages, and (3) activation of a quiescent stem cell, such that the stem cell is activated to divide and the resulting cells maintain the capability of self-renewal and the ability to give rise to all hematopoietic cell lineages. The mpl ligand in the invention initiates at least one mpl-mediated activity, and preferably two or more mpl-mediated activities. Desirably, mpl ligand is thrombopoietin, and more desirably, human thrombopoietin, and most desirably, recombinant human thrombopoietin. The term “mpl ligand” also includes antibodies to the mpl receptor capable of binding to the mpl receptor such that one or more of the above-described mpl-mediated biological actions are initiated. Such antibodies may consist essentially of pooled monoclonal antibodies with different epitopic specificities, or they may be distinct monoclonal antibodies. The term “mpl ligand” further includes mimetic molecules, e.g., small molecules able to bind to the mpl receptor such that one or more of the above-described mpl-mediated biological actions are initiated. Methods known to the art can be utilized to construct libraries of mimetic molecules, and to screen the libraries such that a TPO peptide mimetic is identified having the requisite biological activity.

By “mpl ligand analog” or “TPO agonist analog” is meant a polypeptide that differs from the naturally occurring TPO polypeptide due to the presence of a modification. Known modifications include, but are not limited to, acetylation, acylation, ADP-ribosylation, amidation, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, and sulfation.

By “chimeric TPO polypeptide” is meant a composite polypeptide containing all or part of the coding sequence of thrombopoietin operably linked to all or part of the coding sequence of one or more additional polypeptides (e.g., a growth factor) in which the coding sequences of the thrombopoietin and the one or more additional polypeptides are not naturally found expressed as a single protein.

By “blockade of the TGF-beta pathway” is meant that activation of the TGF-beta pathway is prevented or reduced. Blockade of the TGF-beta pathway occurs, for example, by use of a TGF blocking agent.

By “long-term repopulating stem cell” is meant a hematopoietic or pluripotent stem cell characterized by the ability to give rise to cells that retain the capability of self-renewal, to proliferate and differentiate into cells of all hematopoietic lineages, and to maintain long-term engrafting potential in vivo.

By “signal transduction” is meant the processing of physical or chemical signals from the extracellular environment through the cell membrane and into the cell, and which may occur through one or more of several mechanisms, such as activation/inactivation of enzymes (such as proteases, or other enzymes which may alter phosphorylation patterns or other post-translational modifications), activation of ion channels or intracellular ion stores, effector enzyme activation via guanine nucleotide binding protein intermediates, formation of inositol phosphate, activation or inactivation of adenylyl cyclase, direct activation (or inhibition) of a transcriptional factor and/or activation. A “signaling pathway” refers to the components involved in “signal transduction” of a particular signal into a cell.

By “modulation,” or “modulating” as in “modulating the signal transduction activity of a receptor protein” is meant, in its various grammatical forms, induction and/or potentiation, as well as inhibition and/or downregulation of receptor activity and/or one or more signal transduction pathways downstream of a receptor.

Agonists and antagonists are “receptor effector” molecules that modulate signal transduction via a receptor. Receptor effector molecules are capable of binding to the receptor, though not necessarily at the binding site of the natural ligand or otherwise modulating the activity of the receptor, for example, by influencing the activity of components that regulate the receptor, or which function in the signal transduction pathway initiated by the receptor. Receptor effectors can modulate signal transduction when used alone, i.e. can be surrogate ligands, or can alter signal transduction in the presence of the natural ligand or other known activators, either to enhance or inhibit signaling by the natural ligand. For example, “antagonists” are molecules that block or decrease the signal transduction activity of receptor, e.g., they can competitively, noncompetitively, and/or allosterically inhibit signal transduction from the receptor, whereas “agonists” potentiate, induce or otherwise enhance the signal transduction activity of a receptor.

By “stem cell” or “pluripotent stem cell,” which can be used interchangeably, is meant a stem cell having (1) the ability to give rise to progeny in all defined hematopoietic lineages, and (2) the capability of fully reconstituting a seriously immunocompromised host in all blood cell types and their progeny, including the pluripotent hematopoietic stem cell, by self-renewal. A stem cell or pluripotent stem cell may be identified by expression of the cell surface marker CD34⁺.

Stem cells may be isolated from any known human source of stem cells, including bone marrow, both adult and fetal, mobilized peripheral blood (MPB) and umbilical cord blood. Initially, bone marrow cells may be obtained from a source of bone marrow, including ilium (e.g., from the hip bone via the iliac crest), tibia, femora, spine, or other bone cavities. Other sources of stem cells include embryonic yolk sac, fetal liver, and fetal spleen.

By “Smad” is meant the generally accepted nomenclature for the vertebrate intracellular mediators of TGF-beta signal transduction (see Derynck et al., Cell 87:173, 1996). The Mad (mothers against decapentaplegic) gene in Drosophila and the related Sma genes in Caenorhabditis elegans have been implicated in signal transduction by factors of the TGF-beta family (see Sekelsky et al., Genetics 139:1347-1358, 1995; Savage et al., Proc. Natl. Acad. Sci. USA, 93:790-794, 1996). Related genes have been identified in vertebrates and shown to mediate TGF-beta family signals in these organisms as well. To date, there are eight family members described as full length protein sequences in human, mouse, and/or Xenopus. Because of their diversity and simultaneous identification in different laboratories, the MAD-related products in vertebrates have received different names. In order to facilitate future work and the dissemination of information in this area, it has been proposed to unify the nomenclature of the vertebrate genes and their products by referring to them as “Smad.” This term, a merger of Sma and Mad, differentiates these proteins from unrelated gene products previously called Mad.

### BRIEF DESCRIPTION OF THE DRAWINGS

The figures will now be described.

FIG. 1 is a graph showing the expansion of high proliferative potential (HPP) hematopoietic progenitor cells in the presence of TPO+1D11, an anti-TGF-beta antibody.

FIG. 2 is a graph showing the maintenance in the frequency of HPPs per total cells sampled at weekly intervals for a 3 week period (57, 56, and 67 HPPs per 2,000 cells at weeks 1, 2, and 3 respectively) when cells are cultured in medium containing TPO+1D11.

FIG. 3 is a graph showing that the repopulating potential of long term repopulating cells cultured in TPO alone and TPO+1D11 remained constant on a “per cell” basis under conditions that resulted in a >5,000 fold increase in HPPs over the control at week 3.

### DETAILED DESCRIPTION

Use of a TPO Agonist in Combination with an Exogenously Added TGF-Beta Blocking Agent in the Absence of Exegenously Added SCF for Expansion of Long-Term Repopulating HSC Populations

The present invention is based on the discovery, in part, that ex vivo exposure of HSCs to both TPO and an anti-TGF-beta antibody in the absence of SCF, results in a increase in expansion without differentiation. Exposure of HSCs to either TPO or a TGF-beta blocking agent (e.g., a neutralizing TGF-beta antibody or a TGF-beta antisense nucleic acid) alone results in only a 30-fold increase in expansion, while the combination of TPO and an anti-TGF-beta antibody desirably results in a 20, 40, 60, 80, or 100 fold, more desirably a 200, 400, 600, 800, or 1000 fold, or most desirably a 2000, 3000, 4000, 5000 fold or more increase in expansion. The additive effect of the combination of TPO and a TGF-beta blocking agent on expansion of HSCs has important clinical implications for restoration of hematopoietic capability in subjects in which hematopoietic capability is lost or threatened. Accordingly, the invention features the use of a TPO agonist and a TGF-beta blocking agent (e.g., an anti-TGF-beta antibody or a TGF-beta antisense nucleic acid) for ex vivo expansion of a cell population, e.g., human long-term repopulating hematopoietic stem cells (LTR-HSC). This is particularly useful for re-establishing hematopoietic capability in patients in which native hematopoietic capability has been partially, substantially, or completely compromised.

The present invention can be practiced in several ways. In one embodiment, stem cells from any tissue are removed from a human subject (e.g., from bone marrow), expanded ex vivo by exposure to a TPO agonist in combination with a TGF-beta blocking agent in the absence of exogenously added growth factors (e.g., SCF), and the expanded cells are returned to the patient. If necessary, the process may be repeated to ensure substantial repopulation of the stem cells. The expanded stem cell population returned to the subject retains pluripotent characteristics, e.g., self-renewal and the ability to generate cells of all hematopoietic lineages.

In a second embodiment, a more purified population of HSCs (e.g., long-term repopulating hematopoietic stem cells (LTR-HSC), selected by one of several techniques known in the art, is expanded by exposure to a TPO agonist in combination with a TGF-beta blocking agent in the absence of exogenously added growth factors. In a third embodiment, highly purified HSCs are amplified by growth in culture prior to exposure of a TPO agonist in combination with a TGF-beta blocking agent in the absence of exogenously added growth factors. In all embodiments, it is envisioned that expansion of the HSCs of the invention is at least 10 fold, desirably 20, 40, 60, 80, or 100 fold, more desirably 200, 400, 600, 800, or 1000 fold, or most desirably 2000, 3000, 4000, 5000 fold or more, as compared with the expansion of HSCs described in methods known in the art.

Optionally, in any of the above embodiments, exogenous growth factors selected from Flt3, SCF, VEGF, FGF, and EGF can be added after expansion using a TPO agonist in combination with a TGF-beta blocking agent.

Stems cells are desirably isolated from bone marrow, mobilized peripheral blood, and cord blood. Expansion procedures may be conducted in the presence or absence of stromal cells. Stromal cells may be freshly isolated from bone marrow or from cloned stromal cell lines. Such lines may be human, murine, or porcine. For clinical applications, it is desired to culture the stem cells in the absence of stromal cells. A description of the use of stromal cells can be found in, for example, Peled et al., Exp. Hematol. 24:728-737, 1996, and in U.S. Pat. Nos. 6,255,112, 6,103,522, and 5,879,940. During expansion, TPO may be present only during the initial course of the stem cell growth and expansion, desirably at least 2 to 4 hours, more desirably at least 2 to 24 hours, and most desirably at least about 48 hours to 4 days or more, or is maintained during the entire course of the expansion. During cell expansion, TPO is present in a concentration range of 1-200 ng/ml, more desirably, TPO is present in a concentration range of about 10-100 ng/ml, and most desirably, TPO is present in a concentration range of about 10-50 ng/ml. Alternative TPO agonists can also be used in the methods of the invention and are described in detail below.

A TGF-beta blocking agent, desirably an anti-TGF-beta antibody or a TGF-beta antisense nucleic acid molecule, is also present during the initial course of stem cell growth and expansion, desirably at least 2 to 4 hours, more desirably 24 hours, and most desirably at least about 48 hours to 4 days or more, or is maintained during the entire course of the expansion. During cell expansion using an anti-TGF-beta antibody, the anti-TGF-beta antibody is present in a concentration range of 0.1-200 μg/ml, more desirably in a concentration range of 0.5-100 μg/ml, and most desirably in a concentration range of 0.8-50 μg/ml. A TGF-beta antisense nucleic acid can be used in the methods of the invention at a concentration range of 0.1 to 15 μM, more desirably in a concentration range of 1 to 10 μM, and most desirably in a concentration range of 5 to 8 μM. Alternative TGF-beta blocking agents can also be used in the methods of the invention and are described in detail below.

Optionally, after HSC expansion using a combination of TPO and an anti-TGF-beta antibody or TGF-beta antisense nucleic acid, additional growth factor and cytokines, such as Flt3, SCF, VEGF, FGF, EGF, IL-3, IL-6, IL-11, and IL-12 maybe added. Various in vitro and in vivo tests known to the art may be employed to ensure that the pluripotent capability of the stem cells has been maintained.

Thrombopoietin Agonists

MPL Ligand

Thrombopoietin (TPO) is the ligand of the myeloproliferative leukema (mpl) receptor (Bartley et al., Cell 77:1117, 1994; Kaushansky et al., Nature 369:568, 1994; Lok et al., Nature 269:565, 1994; Kuter et al., Proc. Natl. Acad. Sci. USA 91:11104-11108, 1994; Kuter & Rosenberg, Blood 84:1464, 1994; Wendling et al., Nature 369:571, 1994), and was first identified as the proto-oncogene transduced by the murine myeloproliferative leukemia (MPL) virus (Wendling et al., Blood 73:1161-1167, 1989; Souyri et al., Cell 63:1137-1147, 1990; Vigon et al., Proc. Natl. Acad. Sci. USA 89:5640-5644, 1992; Skoda et al., EMBO J. 12:2645-2653, 1993; Methia et al., Blood 82:1395-1401, 1993). TPO is also referred to as MGDF, or megakaryocyte growth and development factor, mpl ligand, and megapoietin. TPO has been shown to independently stimulate megakaryocyte (MK) progenitor division and MK maturation in vivo and in vitro (Bartley et al., supra; Kuter et al., supra; Kuter & Rosenberg, supra; Wendling et al., supra; de Sauvage et al., Nature 369:533, 1994; Broudy et al., Blood 85:1719-1726, 1995; Lok & Foster, Stem Cells 12:586-598, 1994; Zeigler et al., Blood 84:4045, 1994).

The methods of the present invention utilize the full length TPO polypeptide as well as fragments of this polypeptide. Of particular interest are fragments of at least about 10, 20, 30, or 40 amino acids, more desirably at least about 50, 60, 70, 80, or 90 amino acids, or most desirably about 100, 110, 120, 130, 140 or more amino acids in length that bind to and activate the mpl receptor. Polypeptides of this type are identified by known screening methods. The resultant polypeptides can be tested for the ability to specifically bind the mpl receptor and stimulate cell proliferation via the mpl receptor. Binding is determined by conventional methods, such as that disclosed by Klotz, Science 217:1247-1249, 1982.

In addition to the full length TPO polypeptide, or fragments thereof, the invention also envisions the use of mpl receptor agonists, chimeric TPO polypeptides, and TPO polypeptide analogs and peptide mimetics. TPO polypeptide analogs or peptide mimetics can include a full length TPO polypeptide, or a fragment thereof, with known modifications. Known modifications include, but are not limited to, acetylation, acylation, ADP-ribosylation, amidation, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, prenylation, racemization, selenoylation, and sulfation. Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts, such as Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol 182: 626-646 (1990)) and Rattan et al. (Ann. N.Y. Acad. Sci. 663:48-62 (1992)).

Exemplary TPO agonists of the invention are described in, for example, U.S. Pat. Nos.: 5,795,569; 5,756,083; 5,593,666; 5,932,546; 5,128,449; 6,254,870; 5,989,538; 5,989,537; 5,986,049; 6,083,913; 6,121,238; 6,251,864; 6,066,318; 5,696,250; 5,830,647; 5,869,451; 4,894,440; and 5,155,211.

The invention also envisions the use of an antibody that acts as a mpl receptor agonist. Examples of exemplary mpl receptor agonists can be found in, for example, U.S. Pat. Nos. 6,342,220 and 5,980,893.

Methods for identifying other molecules of the invention that function as TPO agonists, or which function as activators or downstream effectors of the cell signaling pathway involved in TPO receptor activation can be found in, for example, U.S. Pat. Nos. 5,707,803 and 6,312,941.

It will be understood that any agent which exhibits the above-described characteristics finds utility in the methods and compositions of the invention and that the invention is not limited to the specific agents described herein.

Mpl Receptor Signal Transduction Agonists

The method of the invention also contemplates the use of molecules that promote the expansion of HSCs by activating the cell signaling pathway of the mpl receptor. Exemplary molecules are or would activate the Janus kinase (JAK) family of cell signaling molecules (see, for example, U.S. Pat. No. 5,916,792). Agonists of the mpl receptor signaling pathway that are able to activate the JAK/STAT pathway at the level of protein-protein interaction, as well as protein-DNA interaction are included in the methods of the invention.

By way of example, TPO agonists that activate the mpl receptor signal transduction pathway would activate a JAK, for example, JAK1, JAK2, JAK3 and TYK2, which are activated upon ligand binding to the mpl receptor. Activation of JAKs results in phosphorylation of multiple cellular proteins, including the associated cytokine receptors and the JAKs themselves (reviewed in Ihle, Adv. Immunol., 60:1-35, 1995). The phosphorylated tyrosines are potential docking sites for proteins containing specific phosphotyrosine binding domains (e.g. Src homology (SH)2 and phosphotyrosine binding (PTB) domains). Specific signaling proteins are thereby recruited into the cytokine signaling networks. Since JAK2 physically associates with its activating receptors (via the proline-rich region) and is activated within seconds after receptor engagement, it appears that JAK2 activation is an early, perhaps initiating step in signal transduction by mpl receptor-ligand interaction. A number of signaling molecules that appear to be activated by recruitment to JAK2-mpl-receptor complexes include: 1) Shc proteins, which lie upstream of Ras and the mitogen-activated protein (MAP) kinases ERKs 1 and 2, which are implicated in the regulation of cellular growth and/or differentiation. 2) the insulin receptor substrates (IRS) 1 and 2; and 3) the signal transducers and activators of transcription (STAT) 1, 3, 5a, and 5b, which have been implicated as regulators of transcription of a variety of genes. Agonists of the mpl receptor signaling pathway that are able to activate the JAK/STAT pathway at the level of protein-protein interaction or protein-DNA interaction are also included in the methods of the invention.

Transforming Growth Factor-Beta (TGF-beta) Blocking Agents

TGF-beta is a member of a large superfamily of extracellular proteins involved in many aspects of development. TGF-beta is synthesized as a large precursor protein that is proteolytically cleaved to yield the mature protein. TGF-beta binds to and activates a dimeric serine/threonine kinase receptor, which transmits the signal from the cytoplasm to the nucleus of the cell through a network of Smad proteins.

TGF-beta has been shown to directly and reversibly inhibit the initial cell divisions of murine long-term repopulating hematopoietic stem cells (LTR-HSC) in vitro (Sitnicka et al., Blood 88:82-88, 1996; Ploemacher et al., Stem Cells 11:336-347, 1993; Ottmann et al., J. Immunol. 140:2661-2665, 1988; and Cashman et al., Blood 75:96-101, 1990). It follows that blocking the effects of TGF-beta would be expected to promote such initial cell divisions. It will be understood that such blocking may be accomplished using any of a number of methods, e.g., the use of a nucleic acid antisense to TGF-beta or to the TGF-beta receptor, the use of a nucleic acid antisense to a factor involved in TGF-beta upregulation or downstream signaling of the TGF-beta receptor, e.g., TGF-beta signal transduction; the use of a neutralizing antibody that is specifically immunoreactive with TGF-beta or the TGF-beta receptor; or a neutralizing antibody specifically immunoreactive with a factor involved in TGF-beta receptor activation or signaling. An exemplary cell surface factor that directly inhibits TGF-beta includes decorin, a naturally occurring inhibitor of TGF-beta that is a chondroitin-dermatan sulfate proteoglycan and biglycan (see, e.g., Kolb et al., Am. J. Physiol. Lung Cell. Mol. Physiol. 280:L1327-L1334, 2001; Munz et al., Eur. J. Immunol. 29:1032-1040, 1999). By way of example, the biological effect of TGF-beta on HSC may be inhibited by an antisense nucleic acid specific to the TGF-beta receptor ligand, tissue transglutaminase, type I and II TGF-beta receptor subunits, Smad 2, 3, and 4, Rb-1, p15, p21 and p27 signaling components (see, for example, Hatzfeld et al., J. Exp. Med. 174:925-929, 1991; Cardoso et al., Proc. Natl. Acad. Sci. USA 90:8707-8711, 1993; Fortunel et al., Stem Cells 18:102-111, 2000; Fortunel et al., J. Cell Science 111:1867-1875, 1998).

To effectively block the effect of TGF-beta on HSCs, the agent (e.g., antisense nucleic acid or neutralizing antibody) must be specific for TGF-beta, desirably TGF-beta1 or TGF-beta2, and have the ability to promote expansion of HSCs following short term exposure of the cells to the agent in combination with a TPO agonist and in the absence of exogenously provided cytokines. Treatment of HSCs with such an agent is effective to (1) prolong stem cell survival in vitro at 37° C. or 4° C.; (2) promote rapid hematopoietic repopulation following in vivo administration of treated stem cells; (3) induce sustained repopulation following in vivo administration; (4) induce rapid HSC proliferation; (5) induce stem cell proliferation in vitro with a minimal number of cells; and (6) provide for sustained stem cell proliferation in vitro, resulting in the potential to generate various lineages of HSCs.

Upon treatment with such an agent, e.g., an antibody against TGF-beta or a nucleic acid antisense to TGF-beta, in combination with a TPO agonist, treated HSCs maintain the ability to provide long term sustained hematopoietic reconstitution (in vitro and in vivo), and also exhibit the capability of short term in vitro and in vivo repopulation, a quality that untreated stem cells do not possess.

It will be understood that any agent that exhibits the above-described characteristics finds utility in the methods and compositions of the invention and that the invention is not limited to the specific agents described herein.

Additional TGF-Beta Blocking Agents

Autocrine signaling via TGF-beta can prevent enhanced expansion of cells by the methods of the invention. Therefore, prevention of the endogenous expression of TGF-beta by the cells of the invention is also envisioned. An exemplary method to inhibit expression of TGF-beta involves providing IL-10to the cells. IL-10has been shown to stimulate hematopoiesis by decreasing the expression of TGF-beta. See, for example, Van Vlasselaer et al., Clin. Orthop. 313:103-114, 1995; and Van Vlasselaer et al., J. Cell Biol. 124:569-577, 1994. Other methods, described herein (e.g., use of TGF-beta or TGF-beta receptor antisense nucleic acids) are also useful for preventing autocrine signaling via TGF-beta.

The methods of the invention also contemplate the use of soluble TGF-beta receptors that can bind TGF-beta and prevent the interaction of TGF-beta with the TGF-beta receptor (as described in, e.g., U.S. Pat. No. 5,543,143). Alternatively, the methods of the invention can utilize a protease inhibitor that is capable of inactivating a protease responsible for activating a precursor TGF-beta into an active, mature TGF-beta (see also U.S. Pat. No. 5,543,143).

Binding of TGF-beta to the TGF-beta receptor (e.g., the type II receptor) on the cell surface initiates a cascade of signaling events that leads to a myriad of cellular responses. Ligand associated type II receptors recruit type I receptors in a complex. Both the type I and type II TGF-beta receptors are serine/threonine kinases; the ligand-bound type II receptor phosphorylates the type I receptor and activates its kinase activity. Activated type I receptors relay the TGF-beta signal by phosphorylation of its intracellular substrates Smad2 and Smad3. After Smad2 and Smad3 are phosphorylated, they form a complex with Smad4, a common partner involved in the signaling of many TGF-beta related cytokines. The Smad2/3/4 complex moves to the nucleus and functionally collaborates with distinct transcription factors to turn on or off transcription of many TGF-beta-responsive genes that regulate proliferation. Therefore, the invention also envisions the use of molecules, e.g., antisense oligomers, which prevent cell signaling via Smad proteins (e.g., Smad2, Smad3, or Smad4).

The methods of the invention also envision using repressor proteins, for example, the small leucine-rich proteoglycans (SLRPs; e.g., fibromodulin, decorin, biglycan, lumican, PG-Lb, keratocan, and mimecan), EVI-1, ski, and sno, which block the antiproliferative effects of TGF-beta.

Fibromodulin, decorin, biglycan, lumican, PG-Lb, keratocan and mimecan are members of the family of small leucine-rich proteoglycans (SLRPs). They all have core proteins with the leucine-rich repeats (LRR) which usually occupy more than 70% of the core proteins. These proteoglycans are secreted from the cells after synthesis and are found in the extracelluar matrix. The LRR are also found in various other molecules and, therefore, SLRPs form the LRP superfamily with the other LRR-containing molecules. The LRR domain of the SLRPs is flanked by cysteine-rich clusters which may form disulfide bonds. There are four cysteine residues at the amino terminal region and two cysteine residues at the carboxyl terminal side.

Three members of the SLRP family, decorin, biglycan and fibromodulin, have been shown to interact with and block the activity of transforming growth factor beta (TGF-beta; Hildebrand et al., Biochemical J. 302:527-534, 1994). The results of these studies show that these three decorin-type proteoglycans each bind TGF-beta isoforms and that slight differences exist in their binding properties. It has been proposed that they may regulate TGF-beta activities by sequestering TGF-beta into extracellular matrix.

The activity of EVI-1 is described in Hirai et al., Cancer Chemother. Pharmacol. 48:535-540, 2001, Kurokawa et al., Nature 394:92-96, 1998, Izutsu et al., Blood 97:2815-2822, 2001, and in U.S. Pat. No. 6,323,335. EVI-1 is a retinoblastoma (Rb) protein-interacting zinc finger (RIZ) protein whose inappropriate expression leads to leukemic transformation of hematopoietic cells in mice and humans. EVI-1 represses TGF-beta signaling by direct interaction with Smad3 through its first zinc finger motif, thereby blocking the antiproliferative effect of TGF-beta. EVI-1 also represses Smad-induced transcription by recruiting corepressor proteins, for example, C-terminal binding protein (CtBP). EVI-1 associates with CtBP1 through one of the consensus binding motifs, and this association is required for efficient inhibition of TGF-beta signaling.

Overexpression of two other protooncogenes, ski and sno, can also repress TGF-beta signal transduction (see, for example, Liu et al., Cytokine and Growth Factor Reviews 12:1-8, 2001). SKI and SNO directly associate with Smad proteins and block the ability of the Smads to activate expression of many, if not all, TGF-beta-responsive genes.

Exemplary TGF-beta blocking agents of the invention are described in, for example, WO 02/04479; and U.S. Pat. Nos. 5,118,791 and 5,061,786.

Additional TGF-beta antagonists can be identified using methods known to those skilled in the art and as provided in U.S. Pat. No. 6,046,165.

Antibodies

Antibody-based compounds of the invention can also be used and include function-blocking antibodies targeted to, for example, TGF-beta or the TGF-beta receptor, or antibodies that inhibit TGF-beta receptor signaling. A description of anti-TGF-beta antibodies and methods for their use in the expansion of HSCs is found in WO 00/43499 and WO 02/04479. Such antibodies may include, but are not limited to polyclonal, monoclonal, chimeric, humanized, or single chain, or Fab fragments produced by an Fab expression library. Antibodies, i.e., those which block the biological effect of TGF-beta on HSCs, are especially desired. See, e.g., Antibodies: A Laboratory Manual, Cold Spring Harbor Laboratory, 1988.

Human or humanized antibodies are desired for in vivo applications and for treatment of cells to be readministered in vivo due to the lack of potential side effects which often result from an immune response to the antibody itself. Of particular interest in practicing the methods described herein are human monoclonal antibodies specifically immunoreactive with TGF-beta, desirably human monoclonal antibodies specifically immunoreactive with human TGF-beta, which block the biological effects of TGF-beta on HSCs.

An exemplary antibody that acts to antagonize the activation of the TGF-beta receptor is 1D11, described in Dasch et al., J. Immunol. 142:1536-1541, 1989; Wahl et al., J. Exp. Med. 177:225-230, 1993; Miyajima et al., Kidney Int. 58:2301-2313, 2000; and in U.S. Pat. Nos. 5,571,714, 5,772,998, and 5,783,185.

Antisense or Oligonucleotide Analogs

Antisense compounds can be used in the methods of the invention to modulate expression of cell signaling molecules involved in the TGF-beta activation pathway. For example, antisense nucleic acids, which are able to inhibit gene expression with exquisite specificity, are often used by those of ordinary skill to disrupt the function of particular genes. These compounds may include antisense RNA, dsRNA (containing in whole or in part the gene sequence of a target polypeptide), or any other effective nucleic acid-based compound known to be useful for decreasing gene transcription, translation, or expression by those of skill in the art. These compounds can be used to disrupt expression of TGF-beta, the TGF-beta receptor, or TGF-beta signaling molecules. Exemplary antisense oligomers for use in practicing the invention are described in WO 02/04479 and U.S. Pat. Nos. 6,228,648, 6,013,787, 6,013,788, 6,037,142, 6,054,440, and 6,159,694, hereby incorporated fully by reference.

In one embodiment, an antisense nucleic acid effective to block the expression of TGF-beta may be used in the present invention. In another aspect, the antisense oligonucleotide is directed to a region spanning the start codon of an mRNA specific to a factor involved in TGF-beta signal transduction, e.g., VLA-4, tissue transglutaminase, type I or type II TGF-beta receptor subunits, Smad2, Smad3, Smad4, Rb-1, p15, p21, or p27 signaling components.

While antisense oligonucleotides are a desired form of antisense compound, the present invention comprehends other oligomeric antisense compounds, including but not limited to oligonucleotide mimetics such as are described below. The antisense compounds in accordance with this invention desirably include from about 20 to about 30 nucleobases. Particularly desired are antisense oligonucleotides having from about 20 to about 30 nucleobases (i.e., from about 20 to about 30 linked nucleosides).

Specific examples of desired antisense compounds useful in this invention include oligonucleotides containing modified backbones or non-natural internucleoside linkages. Oligonucleotides having modified backbones include those that retain a phosphorus atom in the backbone and those that do not have a phosphorus atom in the backbone. For the purposes of this specification, and as sometimes referenced in the art, modified oligonucleotides that do not have a phosphorus atom in their internucleoside backbone can also be considered to be oligonucleosides.

Desired modified oligonucleotide backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalklyphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′. Various salts, mixed salts and free acid forms are also included.

Representative United States patents that teach the preparation of the above type of phosphorus-containing linkages include, but are not limited to, U.S. Pat. Nos.: 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,625,050; and 5,697,248, each of which is herein incorporated by reference.

Preferred modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above type of oligonucleosides include, but are not limited to, U.S. Pat. Nos.: 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

In other desired oligonucleotide mimetics, both the sugar and the internucleoside linkage, i.e., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, in particular an aminoethylglycine backbone. The nucleobases are retained and are bound directly or indirectly to aza nitrogen atoms of the amide portion of the backbone.

Representative United States patents that teach the preparation of PNA compounds include, but are not limited to, U.S. Pat. Nos.: 5,539,082; 5,714,331; and 5,719,262, each of which is herein incorporated by reference. Further teaching of PNA compounds can be found in Nielsen et al., Science 254:1497, 1991.

Another modification of the oligonucleotides of the invention involves chemically linking to the oligonucleotide one or more moieties or conjugates which enhance the activity, cellular distribution or cellular uptake of the oligonucleotide. Such moieties include but are not limited to lipid moieties such as a cholesterol moiety (Letsinger et al., Proc. Natl. Acad. Sci. USA 86:6553, 1989), cholic acid (Manoharan et al., Bioorg. Med. Chem. Lett. 4:1053, 1994), a thioether, e.g., hexyl-S-tritylthiol (Manoharan et al., Ann. N.Y. Acad. Sci. 660:306, 1992; Manoharan et al., Bioorg. Med. Chem. Let. 3:2765, 1993), a thiocholesterol (Oberhauser et al., Nucl. Acids Res. 20:533, 1992), an aliphatic chain, e.g., dodecandiol or undecyl residues (Saison-Behmoaras et al., EMBO J. 10:111, 1991; Kabanov et al., FEBS Lett. 259:327, 1990; Svinarchuk et al., Biochimie 75:49, 1993), a phospholipid, e.g., dihexadecyl-rac-glycerol or triethylammonium 1,2-di-O-hexadecyl-rac-glycero-3-H-phosphonate (Manoharan et al., Tetrahedron Lett. 36:3651, 1995; Shea et al., Nucl. Acids Res. 18, 1990), a polyamine or a polyethylene glycol chain (Manoharan et al., Nucleosides & Nucleotides 14:969, 1995), or adamantane acetic acid (Manoharan et al., Tetrahedron Lett. 36:3651, 1995), a palmityl moiety (Mishra et al., Biochim. Biophys. Acta 1264:229, 1995), or an octadecylamine or hexylamino-carbonyl-oxycholesterol moiety (Crooke et al., J. Pharmacol. Exp. Ther. 277:923, 1996).

Representative United States patents that teach the preparation of such oligonucleotide conjugates include, but are not limited to, U.S. Pat. Nos.: 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, each of which is herein incorporated by reference.

It is not necessary for all positions in a given compound to be uniformly modified for use in the present invention, and in fact more than one of the aforementioned modifications may be incorporated in a single compound or even at a single nucleoside within an oligonucleotide. The present invention also includes antisense compounds which are chimeric compounds. “Chimeric” antisense compounds or “chimeras,” in the context of this invention, are antisense compounds, particularly oligonucleotides, which contain two or more chemically distinct regions, each made up of at least one monomer unit, i.e., a nucleotide in the case of an oligonucleotide compound. These oligonucleotides typically contain at least one region wherein the oligonucleotide is modified so as to confer upon the oligonucleotide increased resistance to nuclease degradation, increased cellular uptake, and/or increased binding affinity for the target nucleic acid. An additional region of the oligonucleotide may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of oligonucleotide inhibition of gene expression. Consequently, comparable results can often be obtained with shorter oligonucleotides when chimeric oligonucleotides are used, compared to phosphorothioate deoxyoligonucleotides hybridizing to the same target region. Cleavage of the RNA target can be routinely detected by gel electrophoresis and, if necessary, associated nucleic acid hybridization techniques known in the art.

Chimeric antisense compounds of the invention may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures include, but are not limited to, U.S. Pat. Nos.: 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In vivo Administration of a TPO Agonist and a TGF-Beta Blocking Agent

In one aspect, the invention is directed to methods of promoting the expansion of hematopoictic stem cells in vivo in a patient, by administering to the patient a therapeutically effective amount of a TPO agonist (e.g., recombinant human TPO) in combination with a therapeutically effective amount of a TGF-beta blocking agent, contained in a pharmaceutical composition, as described herein.

Administration, to a patient in need thereof, of a combination of a TPO agonist and a TGF-beta blocking agent can be effective for promoting the expansion of HSC, in patients requiring such treatment, by increasing the proliferation potential of HSCs, while preventing differentiation. Methods and techniques for the administration of a TGF-beta blocking agent (e.g., a TGF-beta antisense oligomer) is generally known in the art and is also described in PCT WO 02/04479.

Stem Cells

The cells to be expanded by the methods of the present invention can be isolated from a variety of sources using methods known to one skilled in the art. The cells can be obtained directly from tissues of an individual or from cell lines or by production in vitro from less differentiated precursor cells, e.g., stem or progenitor cells. An example of obtaining precursor cells from less differentiated cells is described in Gilbert, 1991, Developmental Biology, 3rd Edition, Sinauer Associates, Inc., Sunderland Mass. The precursor cells can be from any animal, e.g., mammalian, desirably human, and can be of primary tissue, cell lines, etc. The precursor cells can be of ectodermal, mesodermal or endodermal origin. Any precursor cells that can be obtained and maintained in vitro can potentially be used in accordance with the present invention. In a desired embodiment, the precursor cell is a stem cell. Such stem cells include but are not limited to hematopoietic stem cells (HSC; e.g., LTR-HSCs), stem cells of epithelial tissues such as the skin and the lining of the gut, embryonic heart muscle cells, liver stem cells, kidney stem cells, and neural stem cells (Stemple and Anderson, Cell 71:973-985, 1992). The stem cells can be expanded under cell growth conditions, i.e., conditions that promote proliferation (“mitotic activity”) of the cells. HSCs useful in the methods of the invention are described in, for example, U.S. Pat. Nos. 5,763,197, 5,750,397, 5,716,827, 5,194,108, 5,061,620, and 4,714,680.

The cells of the invention can be selected using an almost infinite variety of different techniques and settings. Many techniques are readily perceived by those skilled in the art. Desired techniques are based on either positive or negative selection or a combination of both techniques. These two techniques, used alone or in combination, allow unwanted cells to be removed from the system and target cells to be harvested whenever desired. A description of positive and negative selection techniques can be found in, for example, U.S. Pat. Nos. 5,925,567, 6,338,942, 6,103,522, 6,117,985, 6,127,135, 6,200,606, 6,342,344, 6,008,040, 5,877,299, 5,814,440, 5,763,266, and 5,677,136.

Proliferation

Hematopoietic stem cells are rare cells that have been identified in fetal bone marrow, umbilical cord blood, adult bone marrow, peripheral blood, liver, and spleen, which are capable of differentiating into each of the myeloerythroid (red blood cells, granulocytes, monocytes), megakaryocyte (platelets) and lymphoid (T-cells, B-cells, and natural killer cells) lineages. In addition, these cells are long-lived, and are capable of producing additional stem cells, a process termed self-renewal. Stem cells initially undergo commitment to lineage restricted progenitor cells, which can be assayed by their ability to form colonies in semisolid media. Progenitor cells are restricted in their ability to undergo multi-lineage differentiation and have lost their ability to self-renew. Progenitor cells eventually differentiate and mature into each of the functional elements of the blood. This maturation process is thought to be modulated by a complex network of regulatory factors.

The ability to promote proliferation of mammalian (e.g., human or murine) hematopoictic progenitor cells ex vivo while maintaining them in a state of relative immature character (i.e., non-differentiated and not committed to become particular types of blood cells or to become cells of a particular hematopoietic lineage) is a feature of the present invention. Proliferation of mammalian HSCs can be induced using a TPO agonist in combination with a TGF-beta blocking agent as described herein. A shortcoming of prior art methods of inducing proliferation of mammalian HSCs in vitro is that those methods often lead to depletion of multi-potential progenitor cells. Thus, although known methods can be used to increase the numbers of HSC and committed blood cell precursors in vitro, prior art methods exhibit limited ability to maintain multi-potential cells among those cells and precursors. As a result, blood cell populations expanded ex vivo using prior methods have limited capacity for restoring the hematopoietic system of a mammal (e.g., an irradiated human) in which the hematopoietic system has been ablated or depleted, either intentionally or as an outcome of a disease.

HSCs for use in the present invention may be derived from human bone marrow, human newborn cord blood, fetal liver, or adult human peripheral blood. HSCs of the invention can be isolated and maintained in vitro using any techniques known to one skilled in the art and include (a) the isolation and establishment of HSC cultures from the future host, or a donor, or (b) the use of previously established long-term HSC cultures, which may be allogeneic or xenogeneic. The HSCs can be made highly enriched or in substantially pure form. This enrichment can be accomplished before, during, or after long-term culturing, and can be done by any techniques known in the art. Long-term cultures of HSCs can be established and maintained using techniques known in the art.

Differentiation

After the precursor cells have been isolated and induced to proliferate according to the methods of the invention, the precursor cells can be maintained in the presence of a TPO agonist in combination with a TGF-beta blocking agent for a length of time (e.g., 2 to 4 hours, 1 to 5 days, 1 to 14 days, or up to 2 months) to inhibit differentiation, such that the cell proliferates to obtain an expanded precursor population according to the present invention. It is desirable that substantially no differentiation of the precursor cells occurs during expansion. The amount of differentiation that occurs can be determined using assays known to one skilled in the art, e.g., those that detect the presence of more differentiated cells by detecting functions associated with a particular stage of differentiation, e.g., expression of differentiation antigens on the cell surface or secretion of proteins associated with a particular state, or ability to generate various cell types, or detecting morphology associated with particular stages of differentiation (see, WO 00/34443 for assays that test the differentiation/functional characteristics of HSCs).

Once the HSC has expanded to the desired numbers, the TPO agonist and/or the TGF-beta blocking agent can be removed (e.g., by separation, dilution), such that at least some of the cells in the expanded population can be induced to differentiate. Optionally, the cells can be differentiated to a terminally differentiated state if the function of that terminally differentiated cell is desired.

Transplantation

The methods of the present invention provide a means to accelerate the recovery of a patient following chemotherapy and/or radiation treatment. An ex vivo expanded HSC composition may serve as a source of HSCs for various cellular and gene therapy applications, for example, the expanded HSC populations of the present invention can be transplanted into a subject in need, for rapid and sustained repopulation of the hematopoietic system.

Such an ex vivo expanded hematopoietic stem cell composition finds utility in both autologous and allogeneic hematopoietic engraftment when readministered to a patient, where the cells are freed of neoplastic cells and graft-versus-host disease (GVHD) can be avoided.

Alternatively, such an ex vivo expanded HSC composition may be used for gene therapy to treat any of a number of diseases. In such cases, HSCs containing a transgene of interest directed toward a particular disease target is prepared in vitro and reinfused into a subject such that the cell type(s) targeted by the disease are repopulated by differentiation of the cells in the HSC composition following reinfusion into the subject. The HSCs can also be genetically modified using gene therapy techniques known to one skilled in the art (see below) to express a desired gene. The modified cells can then be transplanted into a patient for the treatment of disease or injury by any method known in the art that is appropriate for the type of stem cells being transplanted and the transplant site. HSCs can be transplanted intravenously, or they can be transplanted directly into the site of injury or disease.

In addition, treatment of the recipient's own bone marrow or HSCs (e.g., bone marrow extracted from the patient before commencing chemotherapy or radiation therapy), under the conditions described herein, results in an expanded population of HSC and will avoid the current need for immune suppression by minimizing the potential for GVHD following transplantation.

The expanded HSC composition described herein finds utility in therapeutic regimens directed to repopulation of various tissues, including but not limited to liver (Petersen et al., Science 284:1168-1170, 1999) and neuronal tissue (Bjornson et al., Science 283:534-537, 1999).

Methods of introduction of cells for transplantation include but are not limited to intradermal, intramuscular, intraperitoneal, intravenous, subcutaneous, intranasal, and epidural routes. The compounds of the invention may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

Desirably, the expanded precursor cell is originally derived from the subject to which it is administered, i.e., the transplant is autologous.

Gene Therapy

Gene therapy is a fast evolving area of medical and clinical research. Gene therapy encompasses gene correction therapy, and transfer of therapeutic genes and is being applied to treatment of cancer, infectious diseases, multigenic diseases, and acquired diseases.

Exemplary disease targets include, but are not limited to cancer, such as prostate cancer, breast cancer, lung cancer, colorectal cancer, melanoma and leukemia; infectious diseases, such as HIV, monogenic diseases such as CF, hemophilia, phenylketonuria, ADA, familial hypercholesterolemia, and multigenic diseases, such as restenosis, ischemia, and diabetes.

Because HSCs are capable of maintaining their numbers in vivo without exhaustion, can repopulate at least the entire hematopoietic system, and mature blood cells circulate throughout the body where a corrected gene product needs to be delivered or a corrected gene product would cure a particular deficiency (e.g., adenosine deaminase deficiency), HSCs are an optimal vehicle for gene therapy.

Cell transduction is possible in vivo, however, it is simpler and more easily controlled ex vivo or in vitro, rendering ex vivo cultured HSCs extremely useful for therapeutic gene therapy (see, e.g., Buetler, Biol. Blood Marrow Transplant 5:273-276, 1999; Dao, Leukemia 13:1473-1480, 1999; and see generally Morgan et al., Ann. Rev. Biochem. 62:191-217, 1993; Culver et al., Trends Genet. 10:174-178, 1994; and U.S. Pat. No. 5,399,346 (French et al.)).

An exemplary therapeutic gene therapy regimen may include the steps of obtaining a source of HSCs from a subject, HSC enrichment or purification, in vitro or ex vivo HSC expansion by the methods presented herein, transduction of HSC with a vector containing a gene of interest, and reintroduction into a subject. Transduction of the HSCs by gene therapy techniques can be during or after expansion.

The desired subset of primitive human HSCs described herein that respond to a TPO agonist in combination with a TGF-beta blocking agent are long-term repopulating (LTR-HSC) or pluripotent stem cells, characterized by the ability to give rise to cells which retain the capability of self-renewal, and to proliferate and differentiate into cells of all hematopoietic lineages. The cells produced by the methods of the invention can be made recombinant and used in gene therapy. In its broadest sense, gene therapy refers to therapy performed by the administration of a nucleic acid to a subject. The nucleic acid, either directly or indirectly via its encoded protein, mediates a therapeutic effect in the subject. The present invention envisions methods of gene therapy wherein a nucleic acid encoding a protein of therapeutic value (preferably to humans) is introduced into the HSCs, before or after expansion of the cells according to the invention, such that the nucleic acid is expressible by the HSCs and/or their progeny, followed by administration of the recombinant cells to a subject.

The recombinant HSCs of the present invention can be used in any of the methods for gene therapy available in the art. Thus, the nucleic acid introduced into the cells may encode any desired protein, e.g., a protein missing or dysfunctional in a disease or disorder. For general reviews of the methods of gene therapy, see Goldspiel et al., Clinical Pharmacy 12:488-505, 1993; Wu and Wu, Biotherapy 3:87-95, 1991; Tolstoshev, Ann. Rev. Pharmacol. Toxicol. 32:573-596, 1993; Mulligan, Science 260:926-932, 1993; and Morgan and Anderson, Ann. Rev. Biochem. 62:191-217, 1993. Methods commonly known in the art of recombinant DNA technology that can be used are described in Ausubel et al. (eds.), 1993, Current Protocols in Molecular Biology, John Wiley & Sons, NY; and Kriegler, 1990, Gene Transfer and Expression, A Laboratory Manual, Stockton Press, NY.

In an embodiment in which recombinant HSCs are used in gene therapy, a gene whose expression is desired in a patient is introduced into the HSCs such that it is expressible by the cells and/or their progeny, and the recombinant cells are then administered in vivo for therapeutic effect. HSCs or expanded HSCs can be used in any appropriate method of gene therapy, as would be recognized by those in the art upon considering this disclosure. The resulting action of a recombinant HSC or its progeny cells administered to a patient can, for example, lead to the activation or inhibition of a pre-selected gene in the patient, thus leading to improvement of the diseased condition afflicting the patient.

One common method of practicing gene therapy uses viral vectors, for example retroviral vectors (see Miller et al., 1993, Meth. Enzymol. 217:581-599; Boesen et al., Biotherapy 6:291-302, 1994; Clowes et al., J. Clin. Invest. 93:644-651, 1994; Kiem et al., Blood 83:1467-1473, 1994; Salmons and Gunzberg, Human Gene Therapy 4:129-141, 1993; and Grossman and Wilson, Curr. Opin. in Genetics and Devel. 3:110-114, 1993), adenovirus vectors (Kozarsky and Wilson, Current Opinion in Genetics and Development 3:499-503, 1993; Rosenfeld et al., Science 252:431-434, 1991; Rosenfeld et al., Cell 68:143-155, 1992; and Mastrangeli et al., J. Clin. Invest. 91:225-234, 1993), adenovirus-associated vectors (AAV; see, for example, Walsh et al., Proc. Soc. Exp. Biol. Med. 204:289-300, 1993), herpes virus vectors, pox virus vectors; non-viral vectors, for example, naked DNA delivered via liposomes, receptor-mediated delivery, calcium phosphate transfection, lipofection, electroporation, particle bombardment (gene gun), microinjection, cell fusion, chromosome-mediated gene transfer, microcell-mediated gene transfer, spheroplast fusion, or pressure-mediated gene delivery. Numerous techniques are known in the art for the introduction of foreign genes into cells (see e.g., Loeffler and Behr, 1993, Meth. Enzymol. 217:599-618; Cohen et al., 1993, Meth. Enzymol. 217:618-644; Cline, 1985, Pharmac. Ther. 29:69-92) and may be used in accordance with the present invention, provided that the necessary developmental and physiological functions of the recipient cells are not disrupted. Usually, the method of transfer includes the transfer of a selectable marker to the cells. The cells are then placed under selection to isolate those cells that have taken up and are expressing the transferred gene. Those HSCs are then delivered to a patient. The technique should provide for the stable transfer of the gene to the cell, so that the gene is expressible by the cell and preferably heritable and expressible by its cell progeny.

A desired gene can also be introduced intracellularly and incorporated within host precursor cell DNA for expression, by homologous recombination (Koller and Smithies, Proc. Natl. Acad. Sci. USA 86:8932-8935, 1989; Zijlstra et al., Nature 342:435-438, 1989).

Various reports have been presented regarding the efficacy of gene therapy for the treatment of monogeneic diseases, early stage tumors, and cardiovascular disease. (See, e.g., Blaese et al., Science 270:475-480, 1995; Wingo et al., Cancer 82:1197-1207, 1998; Dzao, Keystone Symposium Molecular and Cellular Biology of Gene Therapy, Keystone, Co. Jan. 19-25, 1998; and Isner, Keystone Symposium Molecular and Cellular Biology of Gene Therapy, Keystone, Co. Jan. 19-25, 1998.)

Pharmaceutical Composition

The TPO agonist and TGF-beta blocking agent of the invention may be administered to a patient for in vivo therapy by any method known to one skilled in the art. The TPO agonist and TGF-beta blocking agent may be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Representative United States patents that teach the preparation of such uptake, distribution and/or absorption assisting formulations include, but are not limited to, U.S. Pat. Nos.: 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

The TPO agonist and TGF-beta blocking agent may be administered in the form of a pharmaceutically acceptable salt, ester, or salt of such ester, or any other compound which, upon administration to an animal including a human, is capable of providing (directly or indirectly) the biologically active metabolite or residue thereof.

The pharmaceutical compositions of the present invention may be administered in a number of ways depending upon whether local or systemic treatment is desired and upon the area to be treated. Administration may be topical (including ophthalmic, vaginal, rectal, intranasal, transdermal), oral or parenteral. Parenteral administration includes intravenous drip, continuous infusion, subcutaneous, intraperitoneal or intramuscular injection, pulmonary administration, e.g., by inhalation or insufflation, or intrathecal or intraventricular administration.

Methods well known in the art for making formulations are found, for example, in Remington's Pharmaceutical Sciences (18th edition), ed. A. Gennaro, 1990, Mack Publishing Company, Easton, Pa. Compositions intended for oral use may be prepared in solid or liquid forms according to any method known to the art for the manufacture of pharmaceutical compositions. The compositions may optionally contain sweetening, flavoring, coloring, perfuming, and/or preserving agents in order to provide a more palatable preparation. Solid dosage forms for oral administration include capsules, tablets, pills, powders, and granules. In such solid forms, the active compound is admixed with at least one inert pharmaceutically acceptable carrier or excipient. These may include, for example, inert diluents, such as calcium carbonate, sodium carbonate, lactose, sucrose, starch, calcium phosphate, sodium phosphate, or kaolin. Binding agents, buffering agents, and/or lubricating agents (e.g., magnesium stearate) may also be used. Tablets and pills can additionally be prepared with enteric coatings.

Liquid dosage forms for oral administration include pharmaceutically acceptable emulsions, solutions, suspensions, syrups, and soft gelatin capsules. These forms contain inert diluents commonly used in the art, such as water or an oil medium. Besides such inert diluents, compositions can also include adjuvants, such as wetting agents, emulsifying agents, and suspending agents.

Formulations for parenteral administration include sterile aqueous or non-aqueous solutions, suspensions, or emulsions. Examples of suitable vehicles include propylene glycol, polyethylene glycol, vegetable oils, gelatin, hydrogenated naphalenes, and injectable organic esters, such as ethyl oleate. Such formulations may also contain adjuvants, such as preserving, wetting, emulsifying, and dispersing agents. Biocompatible, biodegradable lactide polymer, lactide/glycolide copolymer, or polyoxyethylene-polyoxypropylene copolymers may be used to control the release of the compounds. Other potentially useful parenteral delivery systems for the polypeptides of the invention include ethylene-vinyl acetate copolymer particles, osmotic pumps, implantable infusion systems, and liposomes.

Liquid formulations can be sterilized by, for example, filtration through a bacteria-retaining filter, by incorporating sterilizing agents into the compositions, or by irradiating or heating the compositions. Alternatively, they can also be manufactured in the form of sterile, solid compositions which can be dissolved in sterile water or some other sterile injectable medium immediately before use.

Compositions for rectal or vaginal administration are desirably suppositories which may contain, in addition to active substances, excipients such as coca butter or a suppository wax. Compositions for nasal or sublingual administration are also prepared with standard excipients known in the art. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops or spray, or as a gel.

The amount of active ingredient in the compositions of the invention can be varied. One skilled in the art will appreciate that the exact individual dosages may be adjusted somewhat depending upon a variety of factors, including the compound being administered, the time of administration, the route of administration, the nature of the formulation, the rate of excretion, the nature of the subject's conditions, and the age, weight, health, and gender of the patient. Generally, dosage levels of between 0.1 μg/kg to 100 mg/kg of body weight are administered daily as a single dose or divided into multiple doses. Desirably, the general dosage range is between 250 μg/kg to 5.0 mg/kg of body weight per day. Wide variations in the needed dosage are to be expected in view of the differing efficiencies of the various routes of administration. For instance, oral administration generally would be expected to require higher dosage levels than administration by intravenous injection. Variations in these dosage levels can be adjusted using standard empirical routines for optimization, which are well known in the art. In general, the precise therapeutically effective dosage will be determined by the attending physician in consideration of the above identified factors.

The candidate compound of the invention can be administered in a sustained release composition, such as those described in, for example, U.S. Pat. No. 5,672,659 and U.S. Pat. No. 5,595,760. The use of immediate or sustained release compositions depends on the type of condition being treated. If the condition consists of an acute or over-acute disorder, a treatment with an immediate release form will be desired over a prolonged release composition. Alternatively, for preventative or long-term treatments, a sustained released composition will generally be desired.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

Compositions and formulations for oral administration include powders or granules, suspensions or solutions in water or non-aqueous media, capsules, sachets or tablets. Thickeners, flavoring agents, diluents, emulsifiers, dispersing aids or binders may be desirable.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Materials and Methods

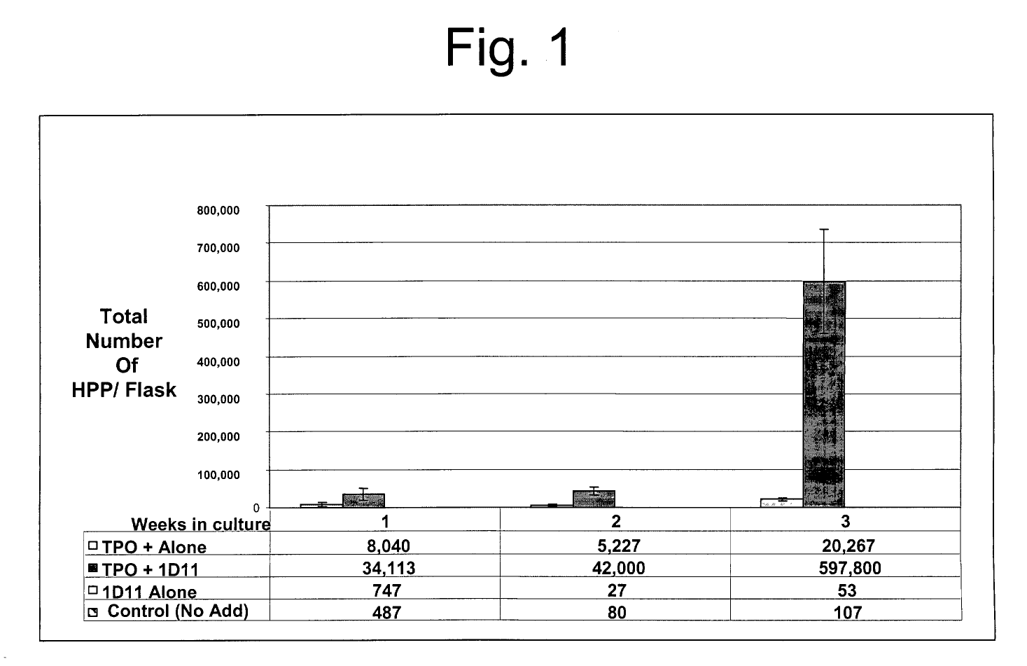

Expansion of HPPs in Culture: Long term murine bone marrow cultures (LTC) were initiated by flushing cells from femurs of male B6.SJL-Ptprc^{a} Pep3^{b}/Boy^{J} (Ly5.1) (CD45.1) (B6.SJL) mice (Jackson Labs, Bar Harbor, Me.) with medium consisting of Fischer's medium (Gibco BRL Life Technologies, Gaithersburg, Md.) supplemented with 20% heat-inactivated defined horse serum (HyClone Laboratories, Logan, Utah), 100 units/ml penicillin-10 μg/ml streptomycin, 2 mM L-glutamine, and 1 μM hydrocortisone succinate (Sigma, St. Louis, Mo.) (Dexter et al., Prog. Clin. Biol. Res. 148:13-33, 1984; Pollack et al., Cell. Immunol. 139:352-362, 1992). Different lots of horse serum varied significantly in their ability to support the production of clonogenic cells (see below). Marrow cells from ten femurs were flushed with complete medium, pooled, and clumps broken up by aspiration through an 18-gauge needle. Cells were directly distributed without washing into 25 cm² tissue culture flasks (B-D Falcon, Lincoln Park, N.J.) at a density corresponding to one femur per flask. Medium was added to a final volume of 8 mL per flask. Where indicated, recombinant mouse thrombopoietin (TPO) (R & D Systems, Minneapolis, Minn.) was added to a final concentration of 10 ng/mL and /or ID11 at 20 ug/mL. Cultures were incubated in a humidified incubator at 37° C. in 5% CO₂ in air. LTC were fed twice weekly beginning after week one by removing 4 mL (for the subsequent four weeks) or 6 mL (thereafter) medium and non-adherent cells, and replacing with an equivalent volume of medium (containing 10 ng/mL TPO and/or 20 ug/mL 1D11). As seen in FIG. 1, the combination of TPO and 1D11 significantly increases the generation of HPPs, in contrast to cultures treated with TPO or 1D11 alone, and the combination promotes the establishment of long-term demi-dilution cultures (LTCs). The net expansion of HPPs in each flask must take into consideration the number of HPPs at time zero plus the number of HPPs that would have been expanded if the demi-dilutions had been retained and then allowed to continue in culture. Using this approach, the theoretical expansion of HPPs over time zero value (approx. 10,000 HPPs per 10,000,000 cells at day zero) is >5,000 fold in 3 weeks.

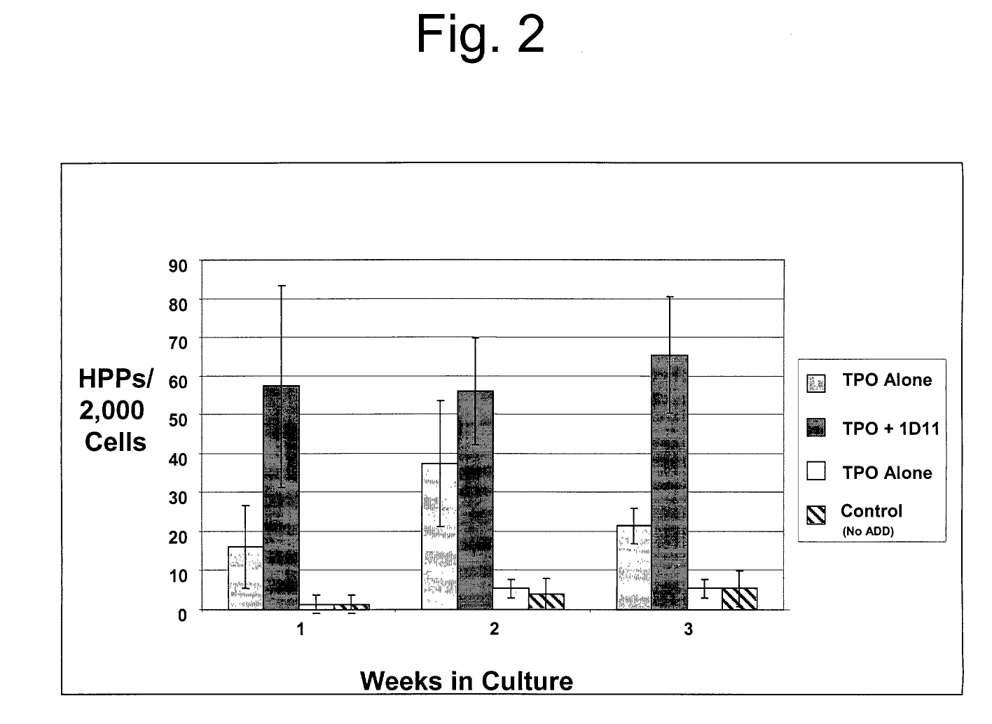

Along with an increase in the absolute number of HPPs in culture (see FIG. 1), a corresponding maintenance in the frequency of HPPs per total cells sampled at weekly intervals for a 3 week period (57, 56, and 67 HPPs per 2,000 cells at weeks 1, 2, and 3 respectively) was observed for cultures containing TPO+1D11 (FIG. 2), suggesting stable population growth without concomitant exhaustion or deviation in surrogate function. Since stem cell content cannot be measured directly, surrogate function refers to an assay method that indirectly measures the presence or function of stem cells. Surrogate function can be confirmed using direct functional assays, for example transplantation.

It will be understood that long term murine bone marrow cultures (LTC) may support the replication of human HSC derived from (1) bone marrow, (2) mobilized peripheral blood, (3) fetal liver, or (4) fetal cord blood using procedures routinely employed by those of skill in the art.

Immunophenotyping of LTC cells: Non-adherent cells from TPO-containing and control LTCs were centrifuged and resuspended in 1% (w/v) bovine serum albumin in Dulbecco's phosphate-buffered saline. Fluorochrome-conjugated monoclonal antibodies to various mouse CD antigens, or biotinylated anti-mouse CD34 and FITC- or PE-conjugated streptavidin (Pharmingen, San Diego, Calif.) were incubated with the cells on ice (1 μg antibody/1-2×10⁵ cells). Cells were washed and analyzed by flow cytometry (FACScan, Becton-Dickinson, Mountain View, Calif.) in the presence of propidium iodide to exclude dead cells.

Clonogenic cell assays: Colony formation assays were performed in double layer soft agar cultures (murine) (Sitnicka et al., Blood 87:4998-5005, 1996). Two thousand to 5,000 cells were added per milliliter of culture and plated in 35 mm dishes. Cultures were incubated for 12 days, and colonies were counted using an inverted microscope. In some experiments, cells were plucked from colonies and their morphology assessed after staining with Giemsa. Cytokines were used at the following concentrations: for HPP-CFC, 50 ng/mL rat SCF, 20 ng/mL human IL-6, and 10 ng/mL mouse IL-3.

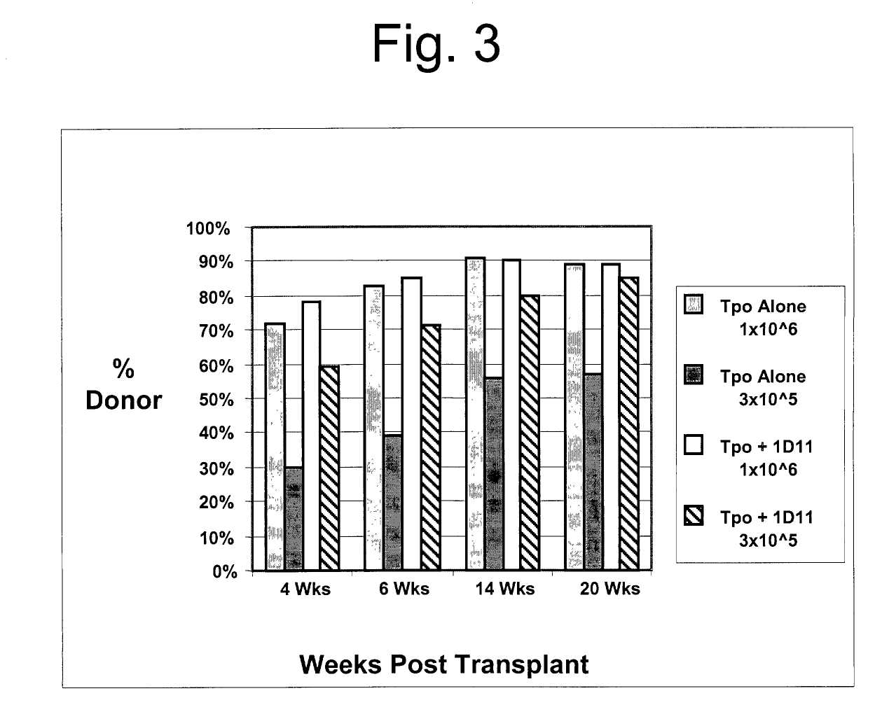

Transplants and competitive repopulation assays. Non-adherent cells from LTCs established from B6.SJL mice (CD45.1) were harvested, washed, and used unfractionated for transplant. For each test sample, 2-10 recipient C57B16 mice (CD45.2) were irradiated (950 rad, ¹³⁷Cesium source) and transplanted by injection via the tail vein with the indicated number of test cells mixed with 4×10⁵ fresh unfractionated CD45.2 marrow cells. In some experiments, cultured cells were transplanted directly without fresh unfractionated bone marrow cells to test the ability of the test cells to rescue the mice from hematopoietic failure instead of the fresh support cells. Animals were maintained in microisolator cages in a specific pathogen free (SPF) facility. Peripheral blood samples were obtained by retro-orbital bleeding 3, 6, 12, and 24 weeks post transplant. Expression of the donor CD45.1 allele and lineage specific antigens was assessed by two-color flow cytometry analysis of peripheral blood leukocytes using directly labeled monoclonal antibodies as described above for cultured cells. The frequency of long-term repopulating units was estimated using the maximum likelihood model that requires limiting dilution cell transplants of the test cells (Taswell, J. Immunol. 126:1614-1619, 1981). As shown in FIG. 3, the long term engraftment potential of mouse HSCs, expanded for 3 weeks under conditions using TPO alone or TPO+1D11, was preserved. This data indicate that the repopulating potential remained constant on a “per cell” basis under conditions that resulted in a >5,000 fold increase in HPPs over the control at week 3 (see FIG. 1). In the mouse/mouse engraftment model, LTRs are verified as present if chimerism increases or remains constant through 12 weeks.

Selection of horse serum lots: Optimal horse serum lots that support the replication of HSC were selected in a two-step process. LTCs were established as described above using TPO alone and HPP assays were performed up to one month of culture (table below). Sera which scored well were selected and set up in fresh LTCs and assayed for repopulating potential of both non-adherent and adherent cells at one month in culture as described above.

TABLE 1
Effect of different serum on the generation
of HPP-CFC in vitro during LTCs with TPO.

| | Total | Total Number | Total Number | Total Number |
|---|---|---|---|---|
| | Number of | of HPP per | of cells/ | of HPP per |
| | cells/flask | flask at 14 | flask at 31 | flask at 31 |
| Serum | at 14 days | days | days | days |
| Hyclone | 0.62 × 10⁶ | 2356±²⁴⁸ | 1.0 × 10⁶ | 2400±⁴⁰⁰ |
| (M.Y.) | | | | |
| AFJ | | | | |
| 5718 | | | | |
| Gibco | 0.94 × 10⁶ | 244±¹¹² | 0.16 × 10⁶ | 0 |
| (E.S.) | | | | |
| Hyclone | 1.26 × 10⁶ | 2772±⁵⁰⁴ | 5.07 × 10⁶ | 24336±⁴⁰⁵⁶ |
| AHA7635 | | | | |
| Hyclone | 2.0 × 10⁶ | nd | 2.86 × 10⁶ | 9152±¹¹⁴⁴ |
| ABA7779 | | | | |
| Gibco | 0.44 × 10⁶ | nd | 0.64 × 10⁶ | 768±⁶⁰⁰ |
| 1016495 | | | | |
| Gibco | 0.90 × 10⁶ | nd | 1.17 × 10⁶ | 328±¹²⁰ |
| 1017003 | | | | |

Preparation and maintenance of murine TPO and/or 1D11 LTC cultures: LTC cultures containing TPO alone or TPO+1D11 were grown in Fisher's medium containing 20% heat inactivated (56° C., 30 min) lot specific horse serum (Hyclone; selected initially by HPP assay followed by the transplantation assay as the ultimate determinant for use of the serum), 100 units/mL penicillin, 10 micrograms/mL streptomycin, 20 mM L-glutamine, and 10⁻⁶ M hydrocortisone (Sigma, catalog #H-0396 (water soluble hydrocortisone)). A stock hydrocortisone solution is made by diluting 9.7 mg/mL in sterile water (equivalent to 1 mg/mL hydrocortisone). The working concentration of hydrocortisone is prepared by adding 180 μL of the stock hydrocortisone solution to 500 mL of medium. Thrombopoietin (TPO) is added to the medium at a concentration up to 20 ng/mL (purified mouse recombinant) and optionally, 1D11 is added up to a concentration of up to 20 ug/mL.

Bone marrow cells are isolated by flushing femurs and tibias with the above complete culture medium and then transfering the cell suspension to T25 flasks (at 10×10⁶ cells/flask) in 8 mL of complete medium per flask. The cells are provided with fresh medium as follows: For the first week, cultures are not disturbed. At the beginning of the second week and following through the fourth week, cells are provided with fresh culture medium twice a week by gently mixing the non-adherent cells by tituration (with the flask canted at ˜45° angle), removing half of the non-adherent cells and medium (4 mL), and replacing with pre-warmed (37° C.) complete medium (described above). During week 5 through week 20+, the bone marrow cell culture medium is replaced as above, except that two-thirds of the non-adherent cells and medium (6 mL) are removed and replaced.

Culture morphology: In the presence of TPO, confluent fibroblast/endothelial stromal layers do not form. Instead, clusters or islands of stroma develop followed by megakaryocyte production and the formation of fibroblast/megakaryocyte clusters with associated primitive hematopoietic cells. Megakaryocytes (CD61/41+) become a predominate non-adherent cell type by 2-3 weeks. Total non-adherent cell production is significantly less in LTC cultures containing TPO than in cultures lacking TPO, however immature cell production is much greater. Megakaryocyte production correlates with HSC production in these cultures.

Assays: We usually use B6SJL congenic mice (CD 45.1+) to establish TPO-LTCs and recipient C57B1/6 (CD45.2+) mice for transplantation assays. In addition, the HPP-CFC assay generally reflects HSC production. Immunophenotyping is useful for quantifying immature cells: c-kit+/Sca-1+ and AA4.1+/ Sca-1+ can predict the number of transplantable cells. It is worth noting that transplantable cells are found both in the adherent (as expected) and non-adherent phase (non-adherent HSC have not been reported previously and may reflect HSC in active cell division).

Preparation of X2 culture medium and X2 agar solution for use in the HPP/Progenitor cell agar assay (mouse): X2 culture media (e.g., 200 mL, final; for both underlayers and overlays) is prepared for use in the HPP/progenitor cell agar assay by adding the following components: 100 mL of fetal calf serum (FCS; selected lot for optimal growth); 100 mL of X3 alpha modified Eagle's medium (MEM; made up as stock from Gibco alpha MEM and frozen as X3 at −20° C. in 120 mL aliquots); and 2 mL penicillin/streptomycin (100× conc.). The solution is warmed to 37° C. and filter sterilized using a 0.2 μm filter.

The X2 agar (1% agar for underlay, 0.6% agar for overlay) is prepared using a stock of Noble Agar or DIFCO® Bacto Agar using sterile, non pyrogenic water (D.W; note: never autoclave the agar). For example, 10 g of DIFCOO® Agar are added to 980 mL D.W. in a sterile 1.5 L flask for a 1% agar solution. The solution is brought to a boil slowly using the #5 setting of a Corning Hotplate and a sterile stirbar. After 20-30 minutes the agar will come to a boil. The agar solution is removed from heat at this point and placed in a 56° C. waterbath for 15-20 minutes to cool. A portion of the 1% agar solution is diluted to 0.6% by taking 232 mL DW+400 mL 1% agar (0.6% Agar). Fifty to seventy-five milliliters of the 0.6% agar and the remaining 1% agar is dispensed into a sterile 125 mL Erlenmeyer flask. Finally, the flasks are set aside at room temperature for storage until use.

Hematopoietic Growth Factors (HGFs) for HPP Assays:

The following hematopoietic growth factors can be used in the HPP assay: recombinant human SCF at a concentration of 50 ng/mL (Amgen, Thousand Oaks, Calif.); recombinant human IL-3 at a concentration of 50 ng/mL (Immunex Corp., Seattle, Wash.); and recombinant human IL-6 at a concentration of 20 ng/mL (Immunex Corp., Seattle, Wash.).

HPP/Progenitor cell assay procedure: The 1% and 0.6% agar solutions are melted using the #5 setting of a Coming Hotplate. The agar is placed in a 37° C. water bath to cool. An aliquot of X2 culture media (˜1 mL/plate; medium can be stored at 4° C. for up to 2-3 weeks) is removed and placed in a 37° C. water bath to warm. The HGF mixture (e.g. one or more selected from SCF, IL-3, IL-6, EPO, TPO, and GM; 0.15 mL/plate; HGF mixture should be 10× strength) is added to the X2 culture media. One part underlayer agar (1%) and 1 part of X2 culture media is mixed to yield a 0.5% final agar concentration. One milliliter of this 0.5% agar solution is added per plate. The agar solution is gently swirled around the plate to spread. The Bone Marrow cells are counted and adjusted to, for example, 300,000/mL, if plating 50,000 cells/dish. See below for more details. Once the under layer has solidified, the 0.6% Agar+X2 media is mixed in a 1:1 volume to yield a 0.3% final agar solution. Cells are always plated in 0.5 mL per 35 mm dish. For example, if 20,000 cells per plate is desired, mix 40,000 cells per mL of the overlay agar (0.5 ml per dish).

The HGF's can be added directly to a batch of underlay agar, but do not allow the agar to solidify. In addition, for best results, the dishes can be placed inside a large ziploc bag with some water or inside a small airtight container pierced with two 18 gauge syringe needles with water dishes, and placed in a low Oxygen (5%) incubator. After a 12 day incubation of the dishes, the number of HPP's (macroclones will have >1 mm dense center) is determined.

All publications and patents mentioned in this specification are herein incorporated by reference to the same extent as if each independent publication or patent application was specifically and individually indicated to be incorporated by reference.

What is claimed is:

1. A method for expanding a desired, relatively undifferentiated cell, said method comprising culturing said cell in a culture medium comprising an exogenously added thrombopoietin (TPO) agonist and lacking exogenously added stem cell factor, under conditions that cause blockade of the transforming growth factor (TGF)-beta pathway.

2. The method of claim 1, wherein said TPO agonist is TPO.

3. The method of claim 1, wherein blockade of the TGF-beta pathway is effected by an exogenously added TGF-beta blocking agent.

4. The method of claim 3, wherein said TGF-beta blocking agent is an anti-TGF-beta antibody.

5. The method of claim 3, wherein said TGF-beta blocking agent is an antisense TGF-beta nucleic acid molecule or an antisense TGF-beta receptor nucleic acid molecule.

6. The method of claim 5, wherein said antisense nucleic acid molecule is RNA.

7. The method of claim 3, wherein said TGF-beta blocking agent an antisense TGF-beta PNA molecule or an antisense TGF-beta receptor PNA molecule.

8. The method of claim 3, wherein said TGF-beta blocking agent is an inhibitor of the TGF-beta receptor signaling pathway.

9. The method of claim 8, wherein said inhibitor of the TGF-beta receptor signaling pathway is an inhibitor of the Smad signaling pathway.

10. The method of claim 9, wherein said inhibitor of the Smad signaling pathway is an inhibitor of Smad2, Smad3, or Smad4.

11. The method of claim 3, wherein said TGF-beta blocking agent is a soluble TGF-beta receptors.

12. The method of claim 3, wherein said TGF-beta blocking agent is a protease inhibitor that inactivates a protease responsible for activating a precursor TGF-beta into an active, mature TGF-beta.

13. The method of claim 3, wherein said TGF-beta blocking agent is a TGF-beta receptor signaling pathway repressor protein.

14. The method of claim 13, wherein said TGF-beta receptor signaling pathway repressor protein is fibromodulin, decorin, biglycan, lumican, PG-Lb, keratocan, mimecan, EVI-1, SKI, or SNO.

15. A method for expanding a desired, relatively undifferentiated cell, said method comprising culturing said cell in a culture medium lacking exogenously added stem cell factor under conditions that promote the activation of the TPO pathway and cause blockade of the TGF-beta pathway.

16. The method of claim 15, wherein activation of the TPO pathway is by activation of the Janus family signaling pathway.

17. The method of claim 16, wherein said activation of the Janus family signaling pathway is by activation of a JAK.

18. The method of claim 17, wherein said JAK is selected from the group consisting of JAK1, JAK2, JAK3, and TYK2.

19. The method of claim 16, wherein said activation of the Janus family signaling pathway is by activation of a STAT.

20. The method of claim 19, wherein said STAT is selected from STAT3 and STAT5.

21. The method of claim 17, wherein said activation of the TPO pathway is by an exogenously added TPO agonist.

22. The method of claim 21, wherein said TPO agonist is TPO.

23. The method of claim 22, wherein said TPO is human TPO.

24. The method of claim 23, wherein said TPO is recombinant human TPO.

25. The method of claim 21, wherein said TPO agonist is a TPO polypeptide analog.

26. The method of claim 21, wherein said TPO agonist is a TPO peptide mimetic.

27. The method of claim 21, wherein said TPO agonist is a chimeric TPO polypeptide.

28. The method of claim 27, wherein said chimeric TPO polypeptide comprises a fragment of TPO and another growth factor.

29. The method of claim 15, wherein said activation of the TPO pathway is by an antibody.

30. The method of claim 29, wherein said antibody activates the mpl receptor.

31. The method of claim 1 or 15, wherein said relatively undifferentiated cell is a hematopoietic stem cell.

32. The method of claim 31, wherein said hematopoietic stem cell is a long-term repopulating hematopoictic stem cell.

33. The method of claim 1 or 15, wherein said relatively undifferentiated cell is a mammalian cell.

34. The method of claim 33, wherein said mammalian cell is a human cell.

35. The method of claim 1 or 15, wherein said culture medium further comprises a growth factor or a cytokine after said expansion.

36. The method of claim 35, wherein said growth factor is selected from Flt3, SCF, VEGF, FGF, and EGF.

37. The method of claim 35, wherein said cytokine is selected from IL-3, IL-6, IL-11, and IL-12.

38. The method of claim 1 or 15, wherein said culturing is for 2 to 4 hours.

39. The method of claim 1 or 15, wherein said culturing is for 1 to 5 days.

40. The method of claim 1 or 15, wherein said culturing is for 1 to 14 days.

41. A relatively undifferentiated cell that has been expanded by culturing said cell in the presence of an exogenously added TPO agonist and in the absence of exogenously added stem cell factor, and under conditions that results in the blockade of the TGF-beta pathway.

42. A relatively undifferentiated cell that has been expanded by culturing said cell under conditions that results in the activation of the TPO pathway and in the absence of exogenously added stem cell factor, and under conditions that results in the blockade of the TGF-beta pathway.

43. A method of restoring or supplementing a compromised immune system in a subject in need thereof, comprising administering a cell that has been expanded by culturing said cell in the presence of an exogenously added TPO agonist and in the absence of exogenously added stem cell factor, and under conditions that results in the blockade of the TGF-beta pathway.

44. A method of restoring or supplementing a compromised immune system in a subject in need thereof, comprising administering a cell that has been expanded by culturing said cell under conditions that results in the activation of the TPO pathway and in the absence of exogenously added stem cell factor, and under conditions that results in the blockade of the TGF-beta pathway.

45. A method of restoring or supplementing a compromised blood forming system in a subject in need thereof, comprising administering a cell that has been expanded by culturing said cell in the presence of an exogenously added TPO agonist and in the absence of exogenously added stem cell factor, and under conditions that results in the blockade of the TGF-beta pathway.

46. A method of restoring or supplementing a compromised blood forming system in a subject in need thereof, comprising administering a cell that has been expanded by culturing said cell under conditions that results in the activation of the TPO pathway and in the absence of exogenously added stem cell factor, and under conditions that results in the blockade of the TGF-beta pathway.

47. The method of claims 43 or 44, wherein said immune system has been compromised by chemotherapy or radiation treatment.

48. The method of claims 45 or 46, wherein said immune system has been compromised by chemotherapy or radiation treatment.

49. The method of any of claims 43, 44, 45, or 46, wherein said subject is a mammal.

50. The method of claim 49, wherein said mammal is a human.

51. The method of any of claims 43, 44, 45, or 46, wherein said administering a cell is by transplantation.

52. The method of any of claims 43, 44, 45, or 46, wherein said cell has been transformed with a recombinant polypeptide.
